(19) United States
(12) Patent Application Publication
(10) Pub. No.: US 2004/0018990 A1
(43) Pub. Date: Jan. 29, 2004

(54) Treatment of obesity

(76) Inventor: Harvey Rosner, New York, NY (US)

Correspondence Address: MUSERLIAN AND LUCAS AND MERCANTI, LLP, 475 PARK AVENUE SOUTH, NEW YORK, NY, 10016, US

(21) Appl. No.: 10/205,120

(22) Filed: Jul. 25, 2002

(51) Int. Cl. A61K 31/704

(52) U.S. Cl. 514/25

(57) ABSTRACT

A method and compositions for controlling weight in human beings.

### STATE OF THE ART

Oral alpha glucosidase inhibitors such as acarbose such as acarbose and miglitol have been described in my U.S. Pat. No. 5,387,361 and U.S. patent application Ser. No. 09/609,847 filed Jun. 5, 2000 as being useful for controlling weight in humans when consumed with carbohydrate containing food. The mechanism of action of alpha glucosidase inhibitors is believed to result from a competitive inhibition of pancreatic amylase and membrane bound intestinal alpha glucoside hydrolase enzymes. Pancreatic alpha amylase hydrolizes complex starches in the lumen of the small intestine. The membrane bound intestinal alpha glucosidases hydrolyze oligo saccharides, trisaccharides and disaccharides to glucose and other monosaccharides in the brush boarder of the small intestines. They have no inhibitory effect against lactase and would therefore not be expected to induce the symptoms of lactose intolerance. The weight gain or loss for an individual is essentially the difference between the weight equivalent of calories absorbed and weight equivalent of the calories burned.

### OBJECTS OF THE INVENTION

It is an object of the invention to improve weight control in humans by ingesting with carbohydrate-containing food an effective amount of alpha glucosidase inhibitors and an amount of honey and/or glucose.

It is another object of the invention to provide a composition of alpha glucosidase inhibitors and glucose and/or honey to control weight in humans.

These and other objects and advantages of the invention will become obvious from the following detailed description.

### THE INVENTION

The invention is directed to a method of controlling weight in humans comprising ingesting with carbohydrate-containing food an amount of an alpha glucosidase inhibitor in an amount sufficient to block the absorption of carbohydrates and an amount of glucose and/or honey before or at the time of ingestion to provide a rise in blood sugar.

It has been found that the alpha glucosidase inhibitors effectively block carbohydrate absorption but one still will continue to eat until blood sugar levels rise sufficiently to act as one of the cut-off switches to suppress the appetite. The ingestion of glucose and/or honey prior to or at the time of ingestion of carbohydrate-containing food acts to increase the blood sugar quickly leading to suppression of appetite. This means less food is consumed. Absorption of glucose is not affected by alpha glucosidase inhibitors.

The ingestion of sugar and/or honey acts as an adjuvant potentiating the effect of the alpha glucosidase inhibitors on weight control. Because of its normal action, the alpha glucosidase inhibitors blunt the effect of the normal blood sugar rise which normally takes place after eating. An increase in blood sugar after eating normally acts as a cut-off switch to eating. The glucose and/or honey produces an increase in blood sugar to help control the appetite and therefore, helps in the control of weight.

Weight gain or loss for an individual is essentially the difference between the weight equivalent of the calories absorbed and the weight equivalent of the calories burned. Acarbose apparently exerts its effect by blocking the absorption of carbohydrates, which means a portion of the carbohydrates consumed at the meal are not absorbed by the body but are excreted by the body rather than absorbed due to the action of acarbose. Acarbose does not affect the digestion of proteins or fats. This lower absorption of carbohydrates results in less weight gain due to the lower consumption of calories.

To be effective, the diet must contain carbohydrates above the monosaccharide level and the use of acarbose for weight control is a major breakthrough in the field of weight control. Treatment with acarbose is a relatively safe method for weight control as the side effects of acarbose are minimal as can be seen from the 2000 Physician's Desk Reference.

Alpha glucosidase inhibitors such as miglitol and acarbose have been used for the treatment of type II diabetes and are marketed under the trademark Precose ® by Bayer in tablet dose of 25, 50 and 100 mg. Acarbose is a prescription drug and the exact dosage for weight control will be determined by the attending physician as a result of the clinical response of the patient. I have determined from my studies that normally, the dosage per meal is dependent upon the amount of carbohydrates in the meal but is usually between 25 to 200 mg, preferably 50 to 150 mg. Acarbose can also be administered in the form of a wafer or can be mixed with the food to reduce the carbohydrate absorption.

The method of weight control can be used to control weight gain, to provide weight loss and for the prevention or treatment of obesity depending upon the amount of carbohydrates consumed at the meals. For example, if a person overindulges during the holiday season, the consumption of acarbose at the meals will lower the amount of weight gained because at least a portion of the carbohydrates are excreted rather than absorbed.

Besides blocking absorption of carbohydrates, glucosidase inhibitors, for example acarbose and miglitol, encourages bacterial fermentation in the digestive tract for more gas production, which gives the feeling of fullness, reducing the amount of food consumed at the meal. This results in a psychological deterrent to over eating. Other glucosidase inhibitors are ethanolic extracts of Mangifera indica bark, aqueous extracts of Touchi which is a traditional Chinese food and natural acylated anthocyanins.

The amount of glucose and/or honey can be easily determined but is usually on the order of 1 teaspoonful to one tablespoonful or 2 tablespoons taken shortly before eating or admixed with the food at the time of consumption of the carbohydrate-containing food.

Various modifications of the method of the invention may be made without departing from the spirit or scope thereof. It is to be understood that the invention is intended to be limited only as defined in the appended claims.

What I claim is:

1. A method of controlling weight in humans comprising ingesting with carbohydrate-containing food an amount of an alpha glucosidase inhibitor in an amount sufficient to block the absorption of carbohydrates and an amount of glucose and/or honey before or at the time of ingestion to provide a rise in blood sugar.

2. The method of claim 1 wherein the human is obese.

3. The method of claim 1 wherein the amount of alpha glucosidase inhibitor used results in weight loss by the human.

4. The method of claim 1 wherein the amount of alpha glucosidase inhibitor used results in reduced weigh gain.

5. The method of claim 1 wherein acarbose is the inhibitor.

6. The method of claim 1 wherein the miglitol is the inhibitor.

7. A composition useful for weight control in humans comprising an amount of alpha glucosidase inhibitor to block carbohydrate absorption and an amount of glucose and/or honey sufficient to increase blood sugar in humans.

8. The composition of claim 7 wherein the inhibitor is acarbose.

9. The composition of claim 7 wherein the inhibitor is miglitol.
